(19) United States
(12) Patent Application Publication
(10) Pub. No.: US 2012/0277866 A1
(43) Pub. Date: Nov. 1, 2012

(54) Angled Bullet-Nose Banana Cage

(76) Inventors: Prakasam Kalluri, Chester, VA (US); Christine Rusbarsky, Raynham, MA (US); Thomas Martin, Raynham, MA (US); Jonathan Howe, Raynham, MA (US)

(21) Appl. No.: 13/096,366

(22) Filed: Apr. 28, 2011

(51) Int. Cl. A61F 2/44

(52) U.S. Cl. 623/17.16

(57) ABSTRACT

A banana-shaped cage adapted for use as an intervertebral fusion cage, wherein the leading direction of the nose of the banana cage is substantially in-line with the angle of the inserter shaft that inserts the cage into the disc space. It has been found that insertion of this cage requires lower insertion forces than the conventional cage whose leading direction substantially follows the arc of the banana curve.

### BACKGROUND OF THE INVENTION

The leading cause of lower back pain arises from rupture or degeneration of lumbar intervertebral discs. Pain in the lower extremities is caused by the compression of spinal nerve roots by a bulging disc, while lower back pain is caused by collapse of the disc and by the adverse effects of articulation weight through a damaged, unstable vertebral joint. One proposed method of managing these problems is to remove the problematic disc and replace it with a porous device that restores disc height and allows for bone growth therethrough for the fusion of the adjacent vertebrae. These devices are commonly called “fusion devices”.

U.S. Pat. No. 4,743,256 (“Brantigan”) discloses an improved surgical method for eliminating spinal back pain caused by ruptured or degenerated vertebral discs by spanning the disc space between adjacent vertebrae with rigid fusion devices, or “cages”, having surfaces facilitating bone ingrowth and bottomed on prepared sites of the vertebrae to integrate the implant with the vertebrae and to provide a permanent weight supporting strut maintaining the disc space. Brantigan teaches that these cages are linearly inserted into the disc space from the posterior side of the spine.

U.S. Pat. No. 6,143,032 (“Schafer”) discloses an intervertebral fusion device having a banana-shape, including leading and trailing walls connected by a convex wall and a concave wall. Although FIG. 4 thereof disclose a leading wall that is thicker than the side walls, the leading wall is not tapered.

U.S. Pat. No. 6,245,108 (“Biscup”) discloses a device comprising a pair of D-shaped cages adapted to fit adjacent one another within the disc space. Each cage has a lordotic anterior-posterior wedge shape, and its curved wall is shorter than its opposite wall so that, in combination, the device provides a dome shape.

U.S. Pat. No. 6,387,130 (“Stone”) discloses providing a plurality of implants which when arranged sequentially produce a banana-shaped device which rests on the anterior half of the disc space. Each implant may have a lordotic shape, as in FIG. 5, and the plurality of implants may be tapered for distraction and lordosis, as in FIG. 6.

PCT Patent Publication Number WO 01/28469 A2 (“Frey”) discloses an intervertebral fusion device having a banana-shape, including leading and trailing walls connected by a convex wall and a concave wall. The Frey cage is inserted non-linearly into the disc space from the posterior side of the spine, so that the leading wall thereof comes to rest on one side of the spine, and the trailing wall comes to rest on the other side of the spine. Because the Frey cage bears against each side of each opposing endplate, only one Frey cage need be used in each surgical procedure.

However, Frey discloses positioning the Frey cage in an essentially lateral orientation about midway between the anterior and posterior ends of the endplates. Because the rim of the endplates provides the most stable bearing surface, the Frey implant must have a width that extends across the width of the endplate. Typically, the width of the such cages is about 32 mm.

PCT Published Patent Application No. WO 01/70144 (“Scolio”) discloses a banana-shaped implant having three vertically-disposed through holes defining two internal planar walls therebetween. The implant further has a concave wall having a plurality of openings disposed therethrough. Lastly, the implant has a lordotic anterior-posterior wedge, as well as front part 3 to rear part 4 angle. FIG. 7 of Scolio discloses a similar implant having two vertically disposed holes. It appears that the geometry of this cage (lordosis and a medial-lateral slope) requires that it be used to support only one half of the disc space, as with the Brantigan cage.

PCT Published Patent Application No. WO 02/17823 (“Kim”) discloses a banana-shaped implant having two vertically-disposed through-holes defining a single internal planar walls therebetween. The implant further has a concave wall and a convex wall, each having a plurality of openings disposed therethrough. The upper and lower bearing surfaces of the implant have pyramidal teeth disposed thereon.

US Published Patent Application 2002/0055781 (“Sazy”) discloses a banana-shaped implant having a mesh structure.

US Published Patent Application 2002/0077700 (“Vargas”) discloses a banana-shaped non-porous implant. Paragraph 0055 of Vargas teaches to set the implant as far anteriorly in the disc space as possible.

U.S. Pat. No. 7,500,991 (Bartish) discloses a banana cage having a sloped nose that extends forward only a moderate distance. That is, the length of the nose is about equal to the width of the anterior wall of the cage. However, in other embodiments, Bartish teaches a banana cage having a bullet nose.

### SUMMARY OF THE INVENTION

Now referring to FIGS. 4A-4C, the insertion of a conventional banana cage typically comprises two steps. In the first step, the cage is initially inserted into the disc space in a orientation that is perpendicular to disc wall. See FIGS. 4A-4B. Once in the disc space, the cage may then be rotated about 90 degrees to take its final orientation. See FIG. 4C. This rotation step is often accomplished by moving the banana cage forward against a J-shaped guide that turns the cage. When the nose of the cage is not pronounced, the direction at which the nose slopes does not appear to affect the insertion mechanics of the cage.

However, it has been unexpectedly found that the insertion mechanics of a banana cage having a pronounced insertion nose appear to be governed by the direction at which the nose slopes forward. That is, when the nose of the cage is “pronounced”, the direction at which the nose slopes significantly affects the insertion mechanics of the cage. This finding allows the cage designer to tailor the direction of the pronounced nose on a banana cage in order to achieve the desired insertion mechanics.

In particular, it has been found that when the direction of the slope of the pronounced nose is in-line with the curvature of the banana cage (see 30 degree nose angle of FIG. 1), the initial insertion of this cage into the disc space requires excessive force. This is because the nose angle differs from the insertion angle, and so provides undesired resistance to the initial insertion of the cage. Moreover, as this type of cage is more apt to move in a path following the banana curve, it is more prone to medially veer off the J-shaped guide during the initial insertion phase.

In contrast, when the angle of the slope of the nose is in-line with the shaft of the inserter (see 0 degree nose angle of FIG. 1), the initial insertion of this cage into the disc space desirably requires much less force, as the direction dictated by linear movement of the the inserter and the direction of the nose are substantially the same. Moreover, this type of cage is more apt to remain on the J-shaped guide during the initial insertion phase, as it has no curving influences. The subsequent rotation of this cage is then easily accomplished by its pivoting during advancement upon the J-shaped guide. For these reasons, the 0 degree cage of FIG. 1 is desirable when using a J-curved guide.

Therefore, in accordance with a first embodiment of the present invention, there is provided an intervertebral fusion device comprising:
a) an anterior wall having a convex horizontal cross section and a width,
b) a posterior wall having a concave horizontal cross section, wherein the anterior and posterior walls define a curving longitudinal axis,
c) leading and trailing end walls between the anterior and posterior walls, the trailing end wall having an insertion hole defining an insertion axis,
d) an upper bearing surface between the anterior and posterior walls having at least one upper opening therethrough, and
e) a lower bearing surface between the anterior and posterior walls having at least one lower opening therethrough,
   wherein the upper and lower openings are in communication to promote bony fusion through the device,
   wherein the leading end wall has a nose having a length that is at least 50% longer than the width of the anterior wall, the nose further having upper and lower sloped portions defining a leading direction,
   wherein the leading direction of the nose is offset from the insertion axis of the insertion hole by no more than 9 degrees.

Preferably, the direction of the slope of the nose is offset from the insertion axis of the insertion hole by no more than 5 degrees.

More preferably, the direction of the slope of the nose is substantially in-line with the insertion axis of the insertion hole.

Also in accordance with the present invention, there is provided an assembly comprising;
i) an intervertebral fusion device comprising:
   a) an anterior wall having a convex horizontal cross section,
   b) a posterior wall having a concave horizontal cross section,
   c) leading and trailing end walls between the anterior and posterior walls, the trailing end wall having an insertion hole,
   d) an upper bearing surface between the anterior and posterior walls having at least one upper opening therethrough, and
   e) a lower bearing surface between the anterior and posterior walls having at least one lower opening therethrough,
      wherein the upper and lower openings are in communication to promote bony fusion through the device,
      wherein the leading end wall has a nose having upper and lower sloped portions defining a leading direction,
ii) an inserter having a shaft defining a longitudinal axis, a proximal handle and a distal connection,
   wherein the distal connection feature of the inserter is received in the insertion hole of the fusion device, and

wherein the leading direction of the nose is offset from the longitudinal axis of the shaft by no more than 9 degrees.

Preferably, the direction of the slope of the nose is offset from the longitudinal axis of the inserter shaft by no more than 5 degrees.

More preferably, the direction of the slope of the nose is substantially in-line with the longitudinal axis of the inserter shaft.

Although it is clear that the pronounced nose whose slope is in-line with the inserter axis is desirable for some situations (such as those using a J-shaped guide), it may also be the case that the pronounced nose whose slope is in-line with the curve of the banana cage (i.e., the 30 degree cage of FIG. 1) may be desirable for other insertion scenarios.

For example, when a J-shaped guide is used, the turning of the cage is relatively easy and so the slope of the pronounced nose can be adjusted to facilitate the initial insertion of the cage. In this case, it is desirable for the banana cage to possess a pronounced nose whose slope is in-line with the inserter. However, if a J-shaped guide is not used (as in some articulating banana cage designs), then it may be much more difficult to rotate or pivot the cage after its initial insertion. In such a case, it may be desirable for the banana cage to possess a pronounced nose whose slope direction is in-line with the banana shape of the cage. Such a slope would impart to the cage a tendency to advance in a curving manner, thereby compensating for the absence of the J-curved guide.

Therefore, in some embodiments, the leading direction of the pronounced nose is offset from the insertion axis of the insertion hole by more than 20 degrees.

Thus, the advantage of this invention is that the pronounced nose allows the cage designer to tailor the direction of the slope of such nose so as to achieve a desired set of insertion mechanics.

### DESCRIPTION OF THE FIGURES

FIG. 1 discloses a cage defining different leading directions, as defined by the sloped portions of the nose.

FIGS. 2A-2E disclose various views of a cage of the present invention.

FIG. 3A-3C discloses a cage of the present invention alongside two commercial cages, wherein the offset between inserter angle and leading direction of the nose of each cage is demonstrated.

FIGS. 4A-4C disclose the insertion of a conventional banana shaped cage into a disc space.

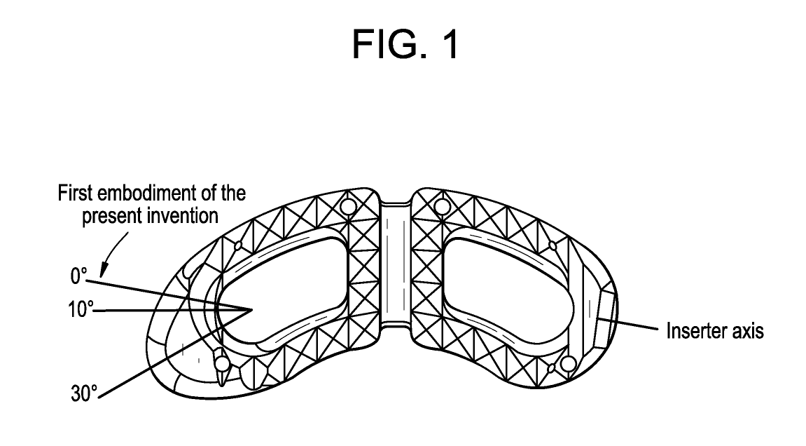

### DETAILED DESCRIPTION OF THE INVENTION

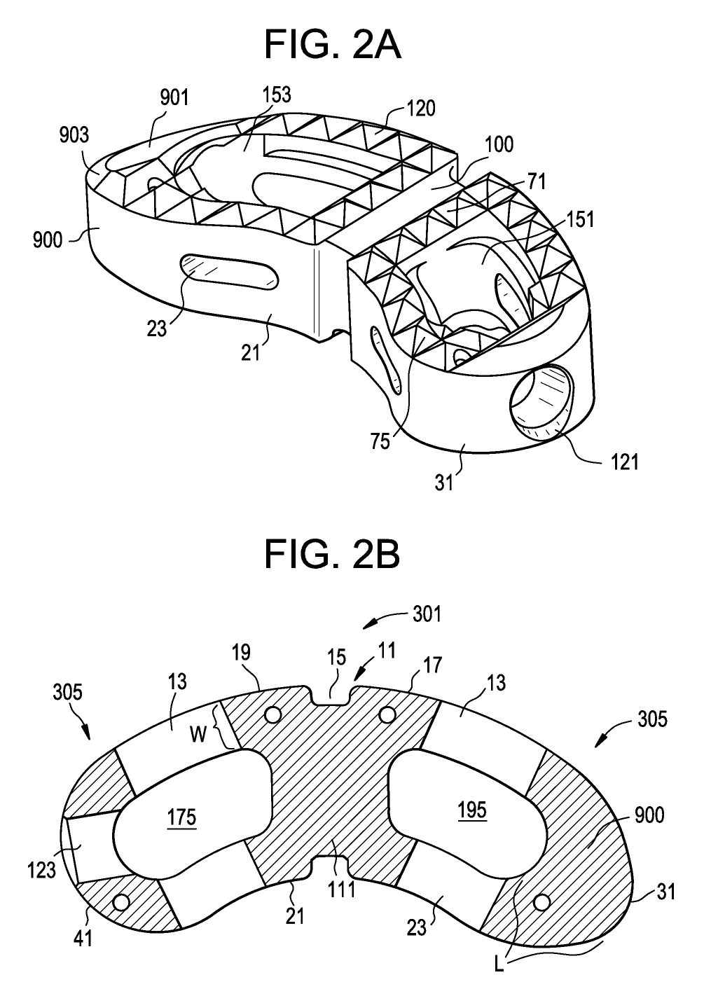

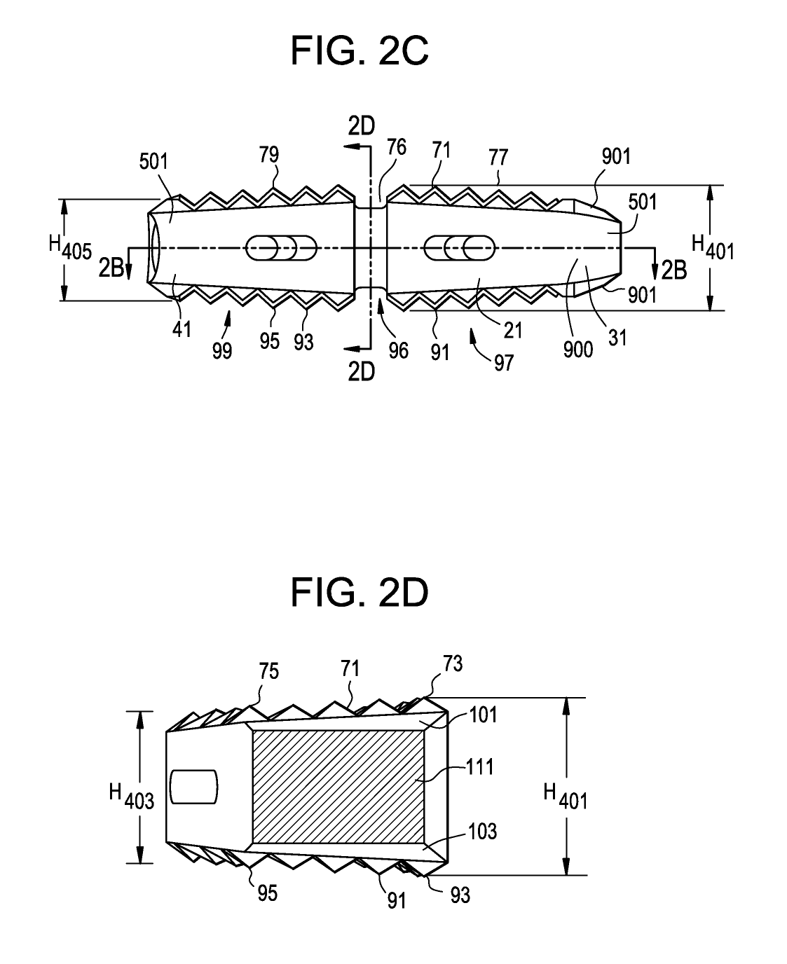

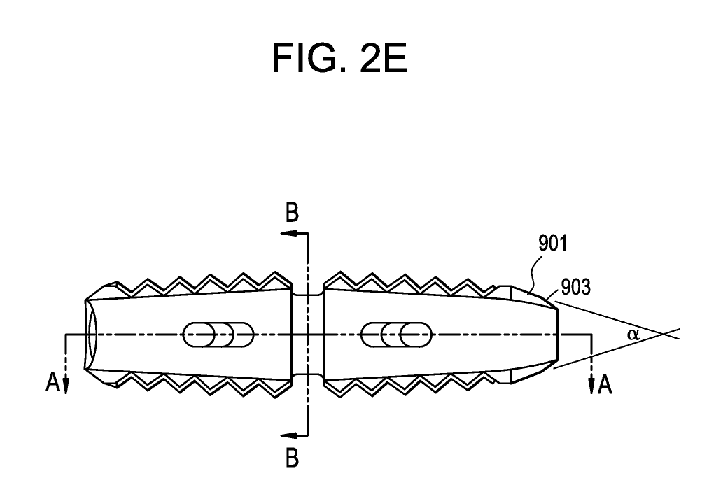

For the purposes of the present invention, the banana cage is considered to have a “pronounced nose” when the nose has a length L that is at least 50% longer than the width W of the anterior wall of the cage, The width W of the anterior wall and the length L of the nose of one particular cage are shown in FIG. 2B.

In this application, the terms “pronounced nose” and “bulleted nose” are used interchangeably.

Preferably, the nose further has upper and lower substantially planar sloped portions, defining a leading direction.

Preferably, the sloped portions of the nose define a total taper angle α therebetween of between 25 and 45 degrees, more preferably between 30 and 40 degrees, more preferably between 35 and 40 degrees. At larger angles, the nose is more blunt and so does not easily distract a collapsed disc space. At smaller angles, the length of the nose must be undesirably long, thereby making the cage unwieldy.

Therefore, in accordance with the present invention, there is provided an intervertebral fusion device comprising:
a) an anterior wall having a convex horizontal cross section and a width,
b) a posterior wall having a concave horizontal cross section, wherein the anterior and posterior walls define a curving longitudinal axis,
c) leading and trailing end walls between the anterior and posterior walls, the trailing end wall having an insertion hole defining an insertion axis,
d) an upper bearing surface between the anterior and posterior walls having at least one upper opening therethrough, and
e) a lower bearing surface between the anterior and posterior walls having at least one lower opening therethrough,
   wherein the upper and lower openings are in communication to promote bony fusion through the device,
   wherein the leading end wall has a nose having a length that is at least 50% longer than the width of the anterior wall, the nose further having upper and lower substantially planar sloped portions defining a leading direction and a total taper angle α therebetween of between 25 and 45 degrees.

In some embodiments, the leading direction of the nose is offset from the insertion axis of the insertion hole by no more than 20 degrees, preferably by no more than 15 degrees, more preferably by no more than 10 degrees. Most preferably, the leading direction of the nose is substantially in-line with the insertion axis of the insertion hole.

In some embodiments, the leading direction is offset from the curving longitudinal axis at the nose tip by at least 15 degrees, preferably by at least 20 degrees, more preferably by at least 25 degrees.

In some embodiments, the leading direction of the nose is offset from the insertion axis of the insertion hole by at least 10 degrees, more preferably at least 20 degrees, more preferably at least 25 degrees, more preferably by about 30 degrees.

In some embodiments, the leading direction is offset from the curving longitudinal axis at the nose tip by no more than 15 degrees, preferably by no more than 10 degrees, preferably by no more than 5 degrees. Most preferably, the leading direction is substantially in-line with the curving longitudinal axis of the cage at the nose tip.

In some embodiments, structural features from the banana cages disclosed in U.S. Pat. No. 7,500,991 (Bartish), the specification of which is incorporated by reference herein in its entirety, may be adopted into the cages of the present invention.

Now referring to FIGS. 2A-2D, there is provided an intervertebral fusion device comprising:
a) an anterior wall 11 having a horizontal cross section having a convex shape,
b) a posterior wall 21 having a horizontal cross section having a concave shape,
c) first 31 and second 41 end walls connecting the anterior and posterior walls, the first end having a bullet nose 900 further having upper and lower sloped portions 901 defining a leading direction,
d) an upper bearing surface 71 having an anterior portion 73 above the anterior wall and a posterior portion 75 above the posterior wall, and
e) a lower bearing surface 91 having an anterior portion 93 below the anterior wall and a posterior portion 95 below the posterior wall,
   wherein the anterior portion of each bearing surface is adapted to bear against the anterior cortical rim of the disc space, and
   wherein the posterior portion of each bearing surface is adapted to bear against the anterior aspect of the disc space.

Preferably, this cage is adapted so that the first end wall is first inserted into the disc space, and the device is then rotated.

Preferably, the anterior wall is convexly curved. More preferably, it is shaped to conform to the shape of the anterior cortical rim of the vertebral endplates. When the anterior wall is so shaped the cage may rest upon the anterior cortical rim of the vertebral endplates and provide support. Typically, the convex curve of the anterior wall is in the form of an arc having a radius of between 15 mm and 25 mm. Such curves allow the cage to be inserted in a non-linear fashion.

In some embodiments, the anterior wall comprises openings 13 adapted to promote bone fusion therethrough.

In some embodiments, these openings have a height and a width, wherein the height of the opening is greater than the width. In this condition, the surrounding material is better able to withstand axial compressive stresses.

In some embodiments, the openings comprises between about 14 areal percent (“areal %”) and about 50 areal % of the anterior wall, preferably between 20 areal % and 30 areal %. In contrast to the Frey structure, whose anterior openings comprises roughly about 70 areal % of the anterior wall, these embodiments have more mass and so provide greater strength to the structure than the Frey structure. This enhanced strength is important because the overall size of the device of the present invention is typically smaller than that of the Frey device.

In some embodiments, the horizontal cross section of the anterior wall comprises a recessed portion 15, thereby defining right 17 and left 19 lateral anterior wall end portions. This recess may be used as an alignment guide within the disc space. It may also allow the implant to be pre-bent (material characteristics permitting) prior to insertion. Lastly, it may provide a port for gripping the implant to effect its removal.

In embodiments particularly advantageous in the lumbar spine, the range of the maximum height of the anterior wall is between about 5 mm and 18 mm, and the range of the maximum thickness of the anterior wall is between about 1 mm and 3 mm.

Preferably, the posterior wall is concave curved. Such curves allow the cage to be inserted in a non-linear fashion. Typically, the concave curve of the posterior wall is in the form of an arc having a radius of between 5 mm and 20 mm.

In some embodiments, the posterior wall comprises openings 23 adapted to promote bone fusion therethrough.

In some embodiments, the openings in the posterior wall in combination comprise between about 14 areal % and about 50 areal % of the anterior wall, preferably between 20 areal % and 30 areal %. In this range, the openings are large enough to allow nutrient transfer through the wall. In contrast to the Frey structure, whose posterior openings comprise about 70 areal % of the posterior wall, these embodiments have more mass and so provide greater strength to the structure than the Frey structure. This enhanced strength is important because the overall size of the device of the present invention is typically smaller than that of the Frey device.

In some embodiments particularly advantageous in the lumbar spine, the maximum height of the posterior wall is between about 5 mm and 18 mm, and the maximum thickness of the posterior wall is between about 1 mm and 3 mm. In some embodiments, the height of the posterior wall is such that it upper surface bears against the upper vertebral endplate. In other embodiments however, the height of the posterior wall is somewhat smaller and the upper and lower surfaces do not bear against the upper and lower endplates endplates.

Preferably, the horizontal cross section of the end wall is convexly curved. Such curves allow for smooth transition between the anterior and posterior walls and facilitate insertion into the disc space. Typically, the concave curve of the posterior wall is in the form of an arc having a radius of between 1.5 mm and 6.5 mm.

In some embodiments, the trailing end wall comprises a feature 121 adapted to engage an insertion instrument. This allows the cage to be inserted essentially lengthwise into a small opening in the posterior side of the disc space, and then rotated so that the anterior wall faces the anterior portion of the disc space. In some embodiments, these features 121 comprise an opening adapted to receive a pusher instrument. In preferred embodiments, the opening is a threaded opening adapted to receive a threaded pusher instrument.

In some embodiments, the openings adapted to receive a pusher instrument are the sole openings in the end walls. This conditions conserves the mass of the end walls, and so provides greater strength to the structure.

The upper and lower surfaces of the cage are adapted to bear against the opposing surfaces of the opposing vertebral bodies defining the disc space. In some embodiments, the upper and lower surfaces are adapted to bear against the endplate portion of the vertebral bodies. In others, channels are cut in the endplates, and these surfaces are adapted to bear against the opposed bone surfaces exposed by these channels.

Preferably, each of the upper and lower surfaces are convexly curved in a lateral-lateral cross section. More preferably, each is shaped to conform to the shape of the opposed surfaces of the vertebral endplates. When the upper and lower surfaces are so shaped, the cage conforms more precisely to the disc space. Typically, the convex curve of the upper and lower surfaces is in the form of an arc having a radius of between 90 mm and 240 mm.

In some embodiments, the upper and lower surfaces comprise openings 175, 195 adapted to promote bone fusion therethrough.

In some embodiments, these openings have a length and a width, wherein the length of the opening is greater than the width of the opening. Since the preferred cages have a long length, in this condition, only a few openings need be filled from the top or bottom in order to desirably fill the cage with graft material.

In some embodiments, the openings comprises between about 30 areal % and about 60 areal % of the upper and lower bearing surfaces. In contrast to the Frey structure, whose upper and lower openings comprises roughly about 70 to 80 areal percent of the upper and lower surfaces, these embodiments of the present invention have more mass and so provide greater strength to the structure and increased resistance to subsidence than the Frey structure. This enhanced strength is important because the overall size of the device of the present invention is typically smaller than that of the Frey device.

In some embodiments, the horizontal cross section of each of the upper and lower surfaces comprises a recessed portion 76, 96, thereby defining right 77, 97 and left 79, 99 upper and lower surface portions. The function of the recessed portion is to from an I-beam structure to help prevent bending during insertion. It can also be used for alignment, for post-operative visualization of the extent of fusion at the endplate-cage interface, and may help resist subsidence.

In embodiments particularly advantageous in the lumbar spine, the maximum length of each of the upper and lower surfaces is between about 20 mm and 30 mm.

In some embodiments, the maximum height 401 of the anterior wall is greater than the maximum height 403 of the posterior wall, such that the upper and lower bearing surfaces provide lordosis. Preferably, the heights are such that the lordosis created is between about 1 degree and about 10 degrees. This range corresponds to the natural physiologic range of lordosis in the lumbar and cervical portions of the spine. In some embodiments, upper and lower surfaces are linearly graded so that the lordotic angle is consistent from the anterior wall to the posterior wall. In other embodiments, the grade can be provided essentially entirely in the anterior wall, or, the grade can be provided essentially entirely in the anterior and end walls.

In some embodiments, the maximum height of the anterior wall is less than the maximum height of the posterior wall, such that the upper and lower bearing surfaces provide kyphosis. Preferably, the heights are such that the kyphosis created is between about 1 degree and about 10 degrees. This range corresponds to the natural physiologic range of kyphosis in the thoracic portion of the spine.

In some embodiments, the anterior wall has a middle portion 301 having a maximum height 401 and lateral end portions 305 each having a maximum height 405, and the maximum height of the middle portion is greater than the maximum height of the lateral end portions. This provides an advantageous doming effect that corresponds to the height of a natural disc space.

In some embodiments, the upper and lower bearing surfaces formed teeth 120 adapted to grip the vertebral endplates and resist cage dislocation. These teeth comprise two angled bearing surface portions that form an angle adapted for gripping the endplates. In some embodiments, the angled bearing surface portions meet to form a sharp point. In other embodiments, a land is disposed between the angled bearing surface portions. The angled nature of the teeth provides a gripping surface that is superior to the grooves formed from essentially parallel surfaces provided in the Frey cage.

In some embodiments, the vertical cross section of each of the upper and lower surfaces comprises a recessed ridge portion 100 thereby defining right 77, 97 and left 79,99 upper and lower surface portions. The function of this ridge is to define a left and a right side of the implant to increase the stability of the implant (like the channel of FIGS. 2A-2E), as well as provide a notch to access the progression of the fusion.

In some embodiments, the openings in the exterior surfaces of the cage extend into the cage to create a chamber 151, 153 therein. This chamber is adapted to hold bone graft material therein and promoting bone fusion therethrough. In some embodiments, the center strut defines dual chambers whose reduced size provides for easier retention of the graft than a single larger chamber.

In preferred embodiments, the cage comprises at least one ridge extending from the anterior portion of the upper bearing surface to the posterior portion of the upper bearing surface. This ridge helps stabilize the cage and increases the mechanical strength of the cage.

In some embodiments, the cage comprises first 101 and second 103 ridges extending from the anterior portion of the upper bearing surface to the posterior portion of the upper bearing surface. The use of two ridges helps prevent medial-lateral rocking of the cage about its midline (as would be the case with a single ridge).

In some embodiments, the first 101 and second 103 ridges are part of a larger internal planar wall 111 extending transversely from the anterior wall to the posterior wall. This internal planar wall effectively splits the cage into right and left portions having right and left graft chambers. This is advantageous when the internal wall is disposed near the centerline of the cage because the smaller chambers can more effectively hold graft material compressed therein than a single large chamber.

In some embodiments, and now referring to FIG. 2E, the nose has a relatively wide distal taper 903 and a relatively narrow proximal taper.

In some embodiments, the relatively narrow proximal taper is defined by the substantially planar sloped portions 901.

In some embodiments, the relatively wide distal taper is defined by a pair of 1 mm chamfers 903.

In some embodiments, and now referring to FIG. 2E, the sloped portions 901 of the nose define a total angle α therebetween of about 38 degrees.

In some embodiments, the nose length is about 6.2 mm.

In some embodiments for a cage that is 7 mm tall, the starting height of the nose is about 3.6 mm.

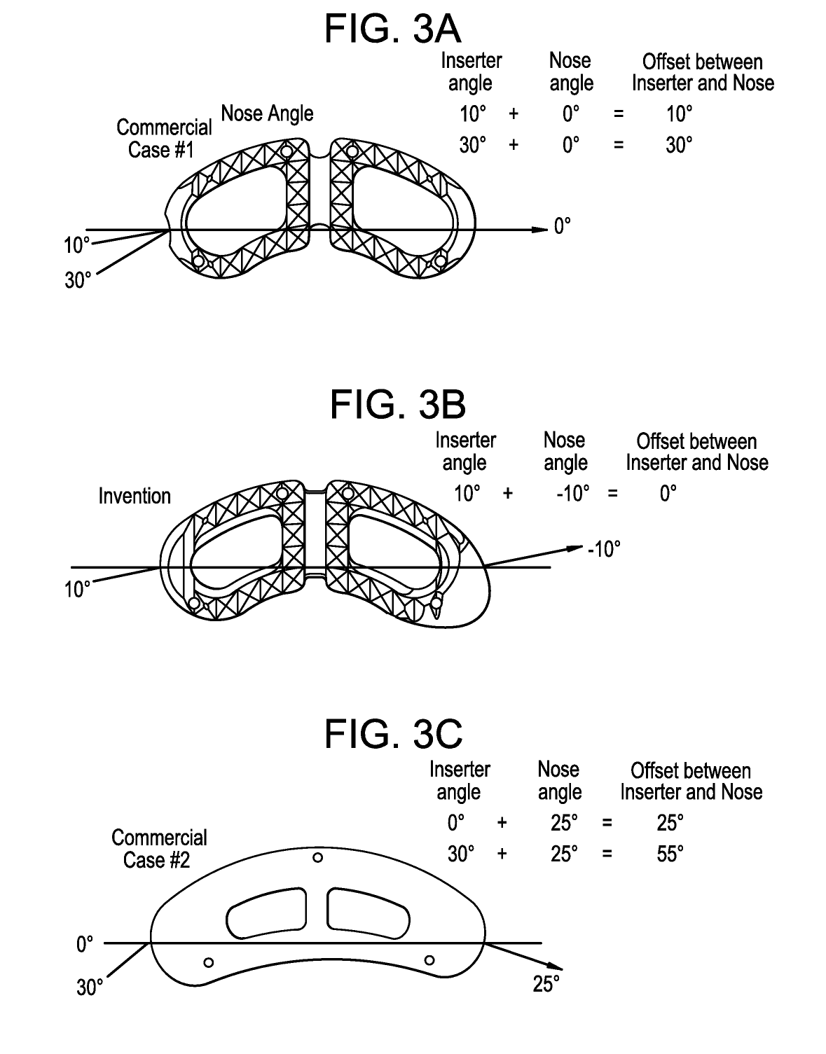

FIGS. 3A-3C discloses a cage of the present invention alongside two commercial cages, wherein the offsets between the leading direction defined by the sloped portions of the nose and the inserter shaft angle are described. The offsets of the commercial cages are at least 10 degrees.

In some embodiments, trials for the cage of the present invention may also include a bullet nose with a taper direction matching that of the implant, thereby allowing the trial to mimic the mechanics of implant insertion as well.

The device of the present invention may be manufactured from any biocompatible material commonly used in interbody fusion procedures.

In some embodiments, the cage is made from a composite comprising:
a) 40-99% polyarylethyl ketone PAEK, and
b) 1-60% carbon fiber
   wherein the polyarylethyl ketone PAEK is selected from the group consisting of polyetherether ketone PEEK, polyether ketone ketone PEKK, polyether ketone ether ketone ketone PEKEKK, and polyether ketone PEK.

Preferably, the carbon fiber is chopped. Preferably, the PAEK and carbon fiber are homogeneously mixed. Preferably, the composite consists essentially of PAEK and carbon fiber. Preferably, the composite comprises 60-80 wt % PAEK and 20-40 wt % carbon fiber, more preferably 65-75 wt % PAEK and 25-35 wt % carbon fiber. In some embodiments, the cage is made from materials used in carbon fibers cages marketed by DePuy AcroMed, Raynham, Mass., USA. In some embodiments, the composite is PEEK-OPTIMA™, available from Invibio of Greenville, N.C.

In other embodiments, the cage is made from a metal such as titanium alloy, such as Ti-6Al-4.

In other embodiments, the cage is made from an allograft material.

In some embodiments, the cage is made from ceramic, preferably a ceramic that can at least partially be resorbed, such as HA or TCP. In other embodiments, the ceramic comprises an oxide such as either alumina or zirconia.

In some embodiments, the cage is made from a polymer, preferably a polymer that can at least partially be resorbed, such as PLA or PLG.

In some embodiments, the cage is provided in a sterile form.

In some embodiments, autologous bone graft material obtained from the iliac crest of the human patient is inserted into the chamber of the cage.

In other embodiments, bone graft material made from allograft particles such as cancellous chips and demineralized bone matrix may be used.

In other embodiments, concentrated osteoinductive materials such as autologous platelet rich plasma or recombinant growth factors may be used.

In other embodiments, concentrated osteogenetic materials such as autologous mesenchymal stem cells (MSCs) or recombinant MSCs may be used.

Preferably, the device of the present invention is placed within the disc space so that the entire device rests within the anterior third of the disc space (ie., the anterior aspect of the disc space). More preferably, the device of the present invention is placed within the disc space so that the entire device rests within the anterior fifth of the disc space, more preferably the anterior eighth of the disc space.

The device of the present invention is intended for non-linear insertion into the intervertebral space through a variety of techniques and approaches, commonly using a single unilateral approach to the disc space.

The design of the implant aids in its safe and efficient insertion into the intervertebral space, and allows for a symmetric single-cage solution to the interbody procedure.

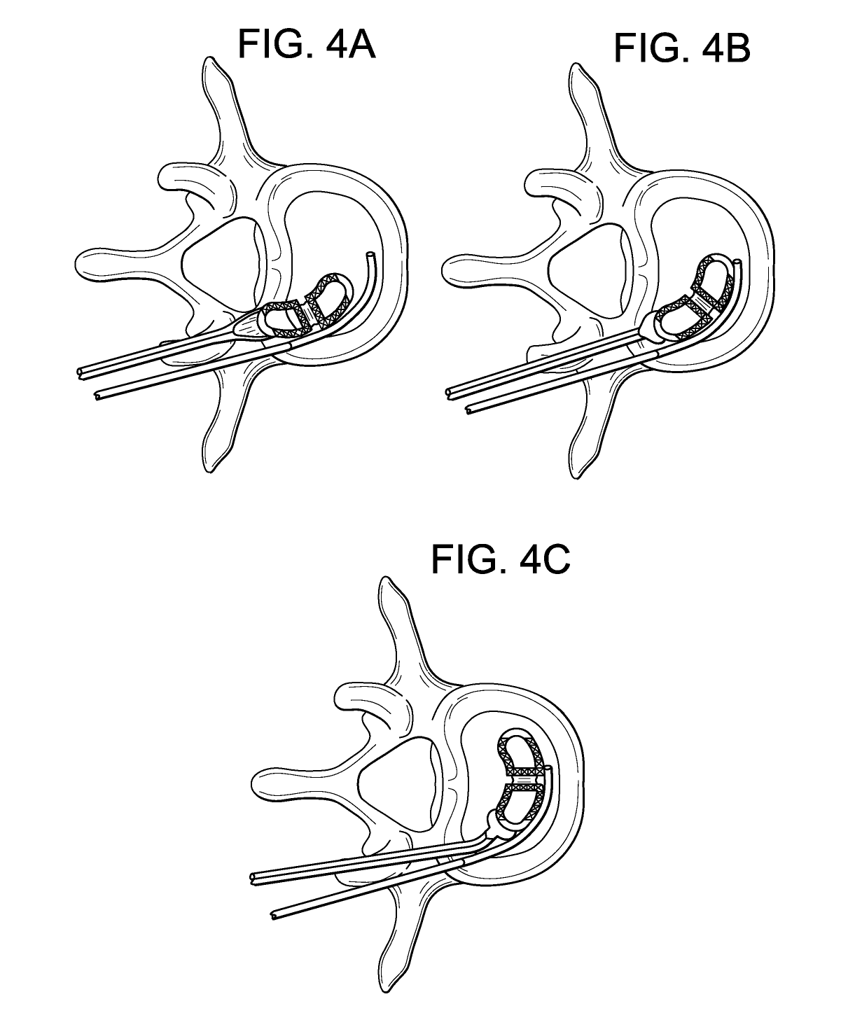

Proceeding in a manner substantially similar to that shown in FIGS. 4A-4C, once the intervertebral disc material has been completely removed and the vertebral endplates prepared with a curved rasp, a rail is inserted into the disc space to act as a guide for selected trials and the implant. The disclosed rail is a curved guide or ramp designed to steer the cage into proper positioning. The curvature of the rail roughly matches the curvature of the anterior wall of the implant.

Next, a trial may be used to determine the appropriate implant size and degree of lordosis.

Next, an inserter is then attached to the implant's insertion hole according to surgical approach and patient anatomy.

The implant is then placed into a cage filler block (not shown) and packed with either autologous bone graft or a substitute.

The implant is then introduced into the disc space using the rail as a guide and back-stop for appropriate implant positioning. Using a mallet if necessary, the implant is inserted nearly into final positioning.

Next, the inserter is detached from the implant due to anatomical considerations. Straight and/or angled impactors are then used to tamp the cage into final positioning using the rail as the guide.

The final positioning of the implant should be in the anterior portion of the disc space and symmetrically located about the medial-lateral midline of the disc space. This will ensure the most stable construct.

Bone graft or a substitute material may then be packed into the remaining posterior half of the disc space to further promote the interbody fusion.

Should it be necessary to remove the implant at any time during the procedure, a remover may be used.

We claim:

1. An intervertebral fusion device comprising:
a) an anterior wall having a convex horizontal cross section and a width,
b) a posterior wall having a concave horizontal cross section, wherein the anterior and posterior walls define a curving longitudinal axis,
c) leading and trailing end walls between the anterior and posterior walls, the trailing end wall having an insertion hole defining an insertion axis,
d) an upper bearing surface between the anterior and posterior walls having at least one upper opening therethrough, and
e) a lower bearing surface between the anterior and posterior walls having at least one lower opening therethrough,
wherein the upper and lower openings are in communication to promote bony fusion through the device,
wherein the leading end wall has a nose having a length that is at least 50% longer than the width of the anterior wall, the nose further having upper and lower sloped portions defining a leading direction,
wherein the leading direction of the nose is offset from the insertion axis of the insertion hole by no more than 9 degrees.

2. The device of claim 1 wherein the leading direction of the nose is offset from the insertion axis of the insertion hole by no more than 5 degrees.

3. The device of claim 1 wherein the leading direction of the nose is substantially in-line with the insertion axis of the insertion hole.

4. An intervertebral fusion device comprising:
a) an anterior wall having a convex horizontal cross section and a width,
b) a posterior wall having a concave horizontal cross section, wherein the anterior and posterior walls define a curving longitudinal axis,
c) leading and trailing end walls between the anterior and posterior walls, the trailing end wall having an insertion hole defining an insertion axis,
d) an upper bearing surface between the anterior and posterior walls having at least one upper opening therethrough, and
e) a lower bearing surface between the anterior and posterior walls having at least one lower opening therethrough,
wherein the upper and lower openings are in communication to promote bony fusion through the device,
wherein the leading end wall has a nose having a length that is at least 50% longer than the width of the anterior wall, the nose further having upper and lower substantially planar sloped portions defining a leading direction and a total taper angle α therebetween of between 25 and 45 degrees.

5. The device of claim 4 wherein the leading direction of the nose is offset from the insertion axis of the insertion hole by no more than 20 degrees.

6. The device of claim 4 wherein the leading direction of the nose is offset from the insertion axis of the insertion hole by no more than 15 degrees.

7. The device of claim 4 wherein the leading direction of the nose is offset from the insertion axis of the insertion hole by no more than 10 degrees.

8. The device of claim 4 wherein the leading direction of the nose is substantially in-line with the insertion axis of the insertion hole.

9. The device of claim 4 wherein the leading direction of the nose is offset from the insertion axis of the insertion hole by at least 10 degrees.

10. The device of claim 4 wherein the leading direction of the nose is offset from the insertion axis of the insertion hole by at least 20 degrees.

11. The device of claim 4 wherein the leading direction of the nose is offset from the insertion axis of the insertion hole by at least 25 degrees.

12. The device of claim 4 wherein the leading direction of the nose is offset from the insertion axis of the insertion hole by about 30 degrees.

13. The device of claim 4 wherein the total taper angle α is between 30 and 40 degrees.

14. The device of claim 4 wherein the total taper angle α is between 35 and 40 degrees.

15. The device of claim 4 wherein the leading direction is offset from the curving longitudinal axis at the nose tip by no more than 15 degrees.

16. The device of claim 4 wherein the leading direction is offset from the curving longitudinal axis at the nose tip by no more than 10 degrees.

17. The device of claim 4 wherein the leading direction is offset from the curving longitudinal axis at the nose tip by no more than 5 degrees.

18. The device of claim 4 wherein the leading direction is substantially parallel to the curving longitudinal axis at the nose tip.

19. The device of claim 4 wherein the leading direction is offset from the curving longitudinal axis at the nose tip by at least 15 degrees.

20. The device of claim 4 wherein the leading direction is offset from the curving longitudinal axis at the nose tip by at least 20 degrees.

21. The device of claim 4 wherein the leading direction is offset from the curving longitudinal axis at the nose tip by at least 25 degrees.

22. An assembly comprising;
i) an intervertebral fusion device comprising:
a) an anterior wall having a convex horizontal cross section,
b) a posterior wall having a concave horizontal cross section,
c) leading and trailing end walls between the anterior and posterior walls, the trailing end wall having an insertion hole,
d) an upper bearing surface between the anterior and posterior walls having at least one upper opening therethrough, and
e) a lower bearing surface between the anterior and posterior walls having at least one lower opening therethrough,
wherein the upper and lower openings are in communication to promote bony fusion through the device,
wherein the leading end wall has a nose having upper and lower sloped portions defining a leading direction,
ii) an inserter having a shaft defining a longitudinal axis, a proximal handle and a distal connection,
wherein the distal connection feature of the inserter is received in the insertion hole of the fusion device, and
wherein the leading direction of the nose is offset from the longitudinal axis of the shaft by no more than 9 degrees.

23. The assembly of claim 22 wherein the leading direction of the nose is offset from the longitudinal axis of the shaft by no more than 5 degrees.

24. The assembly of claim 22 wherein the leading direction of the nose is substantially in-line with the longitudinal axis of the shaft.
